(19) United States
(12) Patent Application Publication
(10) Pub. No.: US 2012/0295335 A1
(43) Pub. Date: Nov. 22, 2012

(54) PREVENTION OF CONTAMINATION OF NUTRIENT FEED RESERVOIRS & FEED LINES IN BIOREACTOR

(75) Inventor: Scott Christopher Jackson, Wilmington, DE (US)

(73) Assignee: E. I. DU PONT DE NEMOURS AND COMPANY, Wilmington, DE (US)

(21) Appl. No.: 13/297,634

(22) Filed: Nov. 16, 2011

Related U.S. Application Data
(60) Provisional application No. 61/414,918, filed on Nov. 18, 2010.

(51) Int. Cl. C12N 1/20; C12N 1/14; C12N 1/16

(52) U.S. Cl. 435/252.5; 435/252.1; 435/253.3; 435/253.2; 435/252.8; 435/255.4; 435/256.1; 435/255.2; 435/256.3

(57) ABSTRACT

A method to prevent contamination of feed line(s) and nutrient feed reservoirs(s) is disclosed. In this method the growth medium which flows from the nutrient feed reservoir through the feed line to the bioreactor contains at least one nutritional component supplied at a concentration which is inhibitory to cell growth. The inhibitory nutritional component in the growth medium prevents back growth of cells from the bioreactor into the feed line(s). The growth medium with inhibitory nutritional component is diluted in the bioreactor.

This application claims the benefit of U.S. Provisional Application 61/414,918, filed Nov. 18, 2010, and is incorporated by reference in its entirety.

### FIELD OF THE INVENTION

The current method relates to the field of microbiology and contamination prevention. Specifically, it relates to a method for prevention of contamination of nutrient feed reservoirs and feed lines providing nutritional components to bioreactors.

### BACKGROUND OF THE INVENTION

Bioreactor systems are commonly used for growth and production of microbial, mammalian and plant cells for various industrial and pharmacological applications. One of the universal problems in operating bioreactor systems, which surpasses mechanical, electrical or instrumentation problems, is the contamination problem which results in process failure. Contamination prevention in bioreactor systems is therefore important to allow uninterrupted production of the desired cells and/or products and to meet manufacturing and production timelines. Additionally, interruption of contaminated bioreactors, clean ups and start ups are costly and can have significant economic impact on the entire process (Junker, B., et al., J. Biosc. Bioeng., 102: 251-2$8, 2006).

Various methods have been used to prevent contamination or spread thereof. Some of these methods depend on application of technologies detrimental to cell growth such as using lethal temperatures, ultraviolet radiation, ionizing radiation and adding chemical inhibitors to the growth medium. For example:

U.S. Pat. No. 4,192,988 disclosed application of electrical heating for heating a sink drain barrier to prevent growth of microorganisms in the drain;

U.S. Pat. No. 3,985,994 disclosed application of an electric heater to heat the interior of the connecting pipe portion between the outlet pipe and the drain pipe of a wash basin to prevent microbes from rising from the drain pipe into the outlet pipe.

Application of ultraviolet light for contamination control in blood products was disclosed in WO01/74407; and U.S. Patent Publication 2008/0142452 disclosed use of UV light in killing microorganisms during water treatment.

Application of intervening physical devices such as air-breaks and filters that physically interrupt penetration of cells and thus prevent contamination has been known and commonly practiced by those knowledgeable in the art (Stanbury, P. R. et al., Principles of Fermentation Technology, 2^{nd} Edition. 1995, Elsevier Sciences Limited, Burlington, Mass.). In the commonly owned application publication (WO 2004101479) filtration was used for removing microbial cells from the product stream of the bioreactor.

Application of bioreactor systems for production of various industrial chemicals and pharmaceutical products, and other applications, has been increasing over the past two decades, thus there is a need for developing effective and economical methods to prevent contamination in these systems without application of intervening physical devices and/or harsh chemicals.

### SUMMARY OF THE INVENTION

The method disclosed herein addresses the need for preventing contamination in a bioreactor system. Specifically, the method teaches prevention of contamination of nutrient feed reservoir(s) and feed line(s) in a bioreactor system during its operation. In the currently disclosed method, no intervening physical device in either the nutrient feed reservoir or the feed line is used for contamination prevention. Rather, contamination prevention is effected through using a growth medium containing one or more inhibitory nutritional component. The bioreactor system disclosed herein comprises a bioreactor, at least one nutrient feed reservoir and at least one feed line. In the disclosed method the growth medium which flows through the feed line to the bioreactor contains at least one nutritional component supplied at a concentration which is inhibitory to cell growth.

In an aspect, the disclosed method for preventing contamination of a feed line and nutrient feed reservoir in a bioreactor system, wherein the bioreactor system comprises a bioreactor, at least one nutrient feed reservoir, and at least one feed line that connects a nutrient feed reservoir to the bioreactor, wherein the method comprises the steps of:

a) adding to the bioreactor an initial charge of cells and inoculum growth medium;

b) filling the feed line with a second growth medium, comprising one or more inhibitory nutritional component, from the nutrient feed reservoir, and

c) adding the second growth medium in the feed line of (b) to the bioreactor of (a) forming a growth medium mixture in the bioreactor; whereby the inhibitory nutritional component of the second growth medium is diluted in the growth medium mixture in the bioreactor and wherein the cells of (a) grow.

In another embodiment the invention provides a bioreactor system comprising a bioreactor, at least one nutrient feed reservoir containing a second growth medium comprising one or more inhibitory nutritional component, and at least one feed line containing said second growth medium that connects the nutrient feed reservoir to the bioreactor.

### BRIEF DESCRIPTION OF THE FIGURE

FIG. 1. Shows a bioreactor system with a single feed line.

### INFORMATION ON DEPOSITED STRAIN

Applicants have made the following biological deposits under the terms of the Budapest Treaty on the International Recognition of the Deposit of Microorganisms for the Purposes of Patent Procedure:

| | International | |
|---|---|---|
| Depositor Identification | Depository | |
| Reference | Designation | Date of Deposit |
| Pseudomonas stutzeri | ATCC No. PTA-11283 | Sep. 9, 2010 |
| BR5311 | | |
| Pseudomonas stutzeri | ATCC No. PTA-8822 | Dec. 4, 2007 |
| LH4:18 | | |
| Pseudomonas stutzeri | ATCC No. PTA-8823 | Dec. 4, 2007 |
| LH4:15 | | |
| Thauera aromatica strain | ATCC No. PTA-9497 | Sep. 17, 2008 |
| AL9:8 | | |
| Arcobacter sp 97AE3-3 | ATCC No. PTA-11410 | Oct. 14, 2010 |
| Arcobacter sp 97AE3-12 | ATCC No. PTA-11409 | Oct. 14, 2010 |

### DETAILED DESCRIPTION

This invention relates to preventing contamination during cell growth in a bioreactor system. More specifically it relates to a method that inhibits back growth of cells from the bioreactor into a feed line and thus prevents contamination of the feed line and a connected nutrient feed reservoir. The term “back growth” as used herein refers to the penetration, of cells from the bioreactor back into a feed line. The term “bioreactor” as used herein refers to a container used to grow cells that produce one or more products for commercial use, which may be the grown cells. The term “feed line”, as used herein, refers to at least a portion of a supply line that carries feed or growth medium from a feed reservoir to the bioreactor. A feed line can comprise several portions that deliver different feed stocks from different sources for mixing and/or subsequent delivery to the bioreactor or can comprise one line that feeds directly from one feed reservoir source. The term “feed reservoir” as used herein refers to a storage container from which growth medium or feed is drawn in order to supply the bioreactor. The term “back growth”, is defined as penetration of cells growing in the bioreactor into the feed line leading to the nutrient feed reservoir.

The present invention may be used for preventing contamination of bioreactor systems using cells of microbial, plant or mammalian origin. The present invention is also particularly useful in bioreactor systems where the growth medium is continuously fed into the bioreactor.

### Apparatus of the Current Method

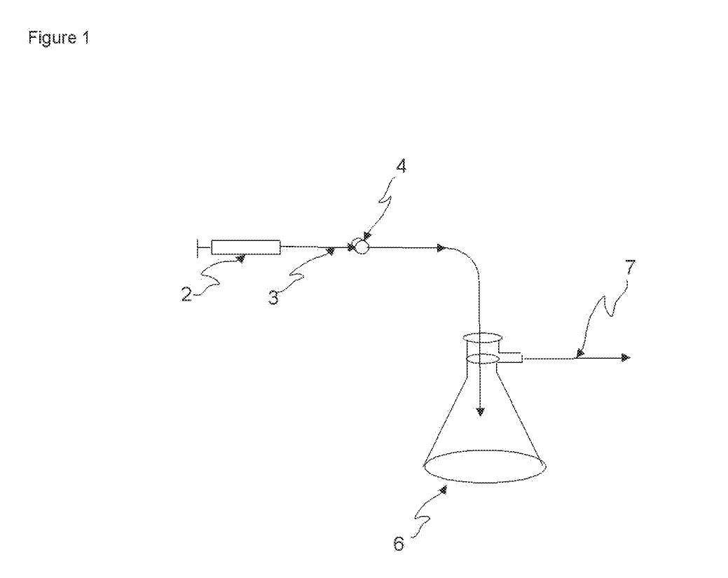

A bioreactor system useful for the present method comprises at least one feed line connecting a nutrient reservoir and a bioreactor. One embodiment of the bioreactor system, with a single feed line, is depicted in FIG. 1 in a laboratory scale design, for illustration. The main components of the system comprise a nutrient feed reservoir (2) which is connected to a nutrient feed line (3) and further to a pump (4) prior to connecting to the bioreactor (6). Effluent from the bioreactor leaves via a side port and an exit line (7). The level of fluid in the bioreactor is shown to be at the level of the side port.

The nutrient feed reservoir (2) can be made of metal, glass, plastic or any other material that can be sterilized and that can maintain its sterility for any length of time. The bioreactor (6) may be of any type that is commonly used in the fermentation industry and is well known to those skilled in the art (Chisti, Y., Chem. Eng. Proc., 88: 80-85, 1992; and Principles of Fermentation Technology, 2^{nd} Edition, 1995, Stanbury. P. F. ed., Elsevier, Burlington, Mass.). The bioreactor may be constructed of steel or glass or any other material suitable for the specific application in mind and can be built in various sizes. The temperature of the bioreactor is regulated by conventional methods, and can be maintained at the temperature optimal for growth of the cells used in the bioreactor. The feed line that is a pipe line and/or tubing connecting the nutrient feed reservoir to the bioreactor can be made of any material suitable for the specific application, which should withstand the pressure used during the process and can be sterilized. The dimensions of the feed line vary depending on the application. The bioreactor system of the current method can be used for either fed-batch or continuous operations. The bioreactor system of the current method can be used for production of a variety of chemicals and/or products for industrial and/or pharmaceutical applications; said product may include cells grown in the bioreactor.

In an embodiment, the bioreactor contains a side arm that acts as a side port and connects to the exit line. The volume of medium and cells used in the bioreactor is such that the liquid level in the bioreactor is at the same level as the side port. Thus any addition of fluid to the bioreactor results in excess fluid flowing out of the side port of the bioreactor into the exit line. In this case, it is desirable that the entering medium be added below the level of the side port to allow mixing of the entering medium with the medium in the bioreactor.

### Growth Media

Growth media useful in the present invention include at least one growth substrate (compounds that supply mass and energy for cell growth); and may include electron acceptors, nitrogen and phosphorus sources, as well as various trace elements such as vitamins and metals that are usually required, in addition to growth substrate and nitrogen sources, for cell growth and activity.

Nutritional components of the growth medium can include the following substances, alone or in combination: one or more carbon source added at greater than 20 parts per million (ppm); one or more electron acceptor for cell growth (under anaerobic growth conditions) added at greater than 50 ppm; a source of nitrogen added at greater than 1 ppm; a source of phosphorous added at greater than 1 ppm; a source of trace nutrients, such, as vitamins and metals, added at greater than 1 ppm.

Useful nutritional components contemplated herein for the growth medium include those containing at least one of the following elements: C, H, O, F, N, S, Mg, Fe, or Ca. Non-limiting examples of such inorganic compounds include: PO₄²⁻, NH₄⁺, NO₂⁻, NO₃⁻, and SO₄⁻ amongst others. In case of microbial cells, growth substrates can include sugars, organic acids, alcohols, proteins, polysaccharides, fats, hydrocarbons or other organic materials known in the art of microbiology to be subject to microbial decomposition. Nutritional components may include major nutrients containing nitrogen and phosphorus (non-limiting, examples can include NaNO₃, KNO₃, NH₄NO₃, Na₂HPO₄, K₂HPO₄, NH₄Cl); vitamins (non-limiting examples may include folic acid, ascorbic acid, and riboflavin); trace elements (non-limiting examples may include B, Zn, Cu, Co, Mg, Mn, Fe, Mo, W, Ni, and Se), buffers for environmental controls; catalysts, including enzymes; and both natural and artificial electron acceptors (non-limiting examples may include SO₄²⁻, NO₃²⁻, Fe⁺³, humic acid, mineral oxides, quinone compounds, CO₂, O₂, and combinations thereof).

In inoculum growth medium, the medium components are those, such as described above, that support growth and productivity of the cells to be grown in the bioreactor. The nutrient components are present in concentrations that support growth of cells used in the bioreactor. One of skill in the art will know the components to use in the inoculum growth medium for a particular cell type.

In the second growth medium of the current method, one or more of the above listed nutrient components is included at a high concentration such that it is inhibitory to cell growth, thus providing an inhibitory nutritional component. Typically high concentration will mean the use of any of these nutrients in excess of 10% w/v. For example many salts, sugars, esters, and alcohols are consumed for growth at low concentrations, but are inhibitory to growth at high concentrations (Microbial Ecology of Foods, V. 1, Silliker et al., (ed.) pages 70-158, 1980 Academic Press, New York, N.Y.). As used herein, “inhibitory nutritional component” means any nutrient in excess of 1 times the concentration at which the nutrient becomes inhibitory (the minimal inhibitory concentration) to the growth of cells used in the bioreactor. Alternatively, these nutrients can be used above 2 times the inhibitory nutrient concentration. In addition, these nutrients can be used above 3 times the inhibitory nutrient concentration or at their solubility limit in water at the nutrient feed temperature, whichever is lower. One of skill in the art will know, or can readily determine, the concentration at which a nutrient component becomes inhibitory to the growth of the cells to be used in the bioreactor.

In one embodiment the carbon source is acetate and in the second growth medium the acetate concentration is greater than 1% making acetate an inhibitory nutrient component. In another embodiment the carbon source is lactate and in the second growth medium the lactate concentration is greater than 1% making acetate an inhibitory nutrient component. In yet another embodiment the electron acceptor is nitrate and in the second growth medium the nitrate concentration is greater than 1% making nitrate an inhibitory nutrient component. Any combination of these components, or combinations with other components, may be used as inhibitory nutritional components.

### Feed to the Bioreactor

In the disclosed method, cells and inoculum growth medium are added as an initial charge to the bioreactor.

In the present method, the feed line that connects the nutrient feed reservoir and the bioreactor is filled with the second growth medium described above, containing one or more inhibitory nutrient component, that is stored in the nutrient feed reservoir. The second growth medium in the feed line is then is added to the bioreactor. Upon addition to the bioreactor, the second growth medium mixes with the cells and medium present in the bioreactor forming a growth medium mixture, thereby diluting the inhibitory nutrient component in the second growth medium. Dilution and consumption of the inhibitory nutrient component by the cells growing in the bioreactor bring the concentration of the inhibitory nutrient component to a level that supports growth of the cells in the bioreactor. Typically following dilution and consumption in the bioreactor an inhibitory nutrient, is reduced to a level that is less than about 10% of the concentration of the inhibitory nutrient in the second growth medium. The inhibitory nutrient may be reduced to a level that is less than about 10%, 9%, 8%, 7%, 6%, 5%, 4%, 3%, 2%, 1° k, or less of the concentration of the inhibitory nutrient in the second growth medium. However, the inhibitory nutrient remains at a concentration that supports growth of the cells in the bioreactor.

In an embodiment, the second growth medium is continuously fed into the bioreactor and the concentration of the carbon source, such as acetate, in the growth medium in the nutrient feed reservoir is maintained following a period of time, such as 28 days, of continuous growth of cells, such as Pseudomonas stutzeri BR5311 (ATCC PTA-11283), in the bioreactor.

In other embodiments, other carbon sources such as sucrose or other sugars, or any of the citric acid cycle compounds suitable for microbial growth can be used in the second growth medium.

### Cells Useful for the Present Invention

Cells including microbes, mammalian cells or plant cells, which are useful for the current disclosure, may be used free or immobilized on inert, insoluble materials such as glass beads, calcium alginate, silica fines or molecular sieves.

### Microbial Cells

For the purposes of the current disclosure, any microbial cells (bacteria, fungi and yeasts), that may be Gram positive or Gram negative, and comprising classes of strict aerobes, facultative aerobes, obligate anaerobes, and denitrifiers that are amenable to growth in bioreactors can be used. The bioreactor can comprise only one particular species or can comprise two or more species of the same genera or a combination of different genera of microbes, including with one or more species of each.

Examples of microbial cells useful far the disclosed method include, but are not limited to Comamonas, Fusibacter, Marinobacterium, Petrotoga, Shewanella, Pseudomonas, Vibrio, Thauera, Microbulbife, Corynebacteria, Achromobacter, Acinetobacter, Arthrobacter, Bacilli, Nocardia, Actinomycetes, Escherichia, Salmonella, Arthrobacter, Acetobacter, Candida, Aspergilli, Saccharomyces, Zymomonas and Penicillium.

The present invention is particularly useful when cells grown in a bioreactor have properties promoting contamination of feeding medium stored in a connected reservoir. Properties of cells including motility and formation of biofilms may promote back growth into feeding lines and medium reservoirs connected to a bioreactor in which the cells are grown.

In particular, cells that are useful for microbially enhanced oil recovery (MEOR) may have one or both of these properties. The present bioreactor system and method may be used to grow cells for use in MEOR processes, an example of which is described in commonly owned and co-pending US Patent Application Publication #2011/0030956, which is herein incorporated by reference. The grown cells are used as an inoculum that is introduced into an oil reservoir to enhance secondary oil recovery.

In one embodiment Pseudomonas aeruginosa cells can be used in the bioreactor. In another embodiment Shewanella putrefaciens LH4:18 (ATCC PTA-8822) cells can be used in the bioreactor. In another embodiment Pseudomonas stutzeri LH4:15 (ATCC PTA-8823) cells can be used in the bioreactor. In another embodiment Pseudomonas stutzeri strain BR5311 (ATCC PTA-11283) cells can be used in the bioreactor. In another embodiment Arcobacter sp. strain 97AE3-12 (ATCC PTA-11409) or strain 97AE3-3 (ATCC PTA-11410) cells can be used in the bioreactor. In another embodiment Thauera aromatica (ATCC PTA-9497) cells can be used in the bioreactor.

Techniques and various suitable media for growth and maintenance of aerobic and anaerobic microbial cells are well known in the art and have been described in “Manual of Industrial Microbiology and Biotechnology” (A. L. Demain and N. A. Solomon, ASM Press, Washington, D.C., 1986) and “Isolation of Biotechnological Organisms from Nature”, (Labeda, D. P. ed. p 117-140, McGraw-Hill Publishers, 1990).

### EXAMPLES

The present invention is further defined in the following Example. It should be understood that the Example, while indicating a preferred embodiment of the invention, is given by way of illustration only. From the above discussion and this Example, one skilled in the art can ascertain the essential characteristics of this invention, and make various changes and modifications to the invention to adapt it to various uses and conditions.

### General Methods and Apparatus

Optical density was measured using a Beckman Coulter DU 7500 with a one centimeter path length cell as is well known in the art.

Miller's Lauria Bertani (LB) growth medium was purchased from Mediatech, Manassas, Va.

### Growth Media

Inoculum Growth Medium for Pseudomonas stutzeri

The filter-sterilized inoculum growth medium for Pseudomonas stutzeri had the following composition: tap water, 495 milliliter (ml); ammonium lactate, 2.5 ml of a 20 percent weight for weight (% w/w) solution; yeast extract, 0.05 grams (g); NaNO₃, 1.0 g; NaCl, 5 g, NH₄Cl, 0.05 g; H₂PO₄, 0.01 g; pH 6.5, fill to 500 ml with tap water.

Inhibitory Growth Medium for Pseudomonas stutzeri

The second growth medium containing inhibitory nutrient component has the following composition: 10 g of filter sterilized tap water was used to dissolve: NaAcetate, 1.53 g; NaNO₃, 3.0 g; Na₃(PO₄), 0.16 g; NH₄Cl, 0.26 g; yeast extract, 0.13 g; and the pH was adjusted to 6.5. The concentration of nitrate is inhibitory, due to nitrate concentration becoming inhibitory at greater than 1 wt %. In addition, the concentration of acetate is inhibitory, due to acetate concentration becoming inhibitory at greater than 1%.

### Ion Chromatography

To quantitate nitrate, nitrite, acetate and lactate ions in aqueous media, a DX-120 chromatography unit (Dionex, Banockburn, Ill.) is used. Ion exchange is accomplished on an AS14A anion exchange column using an isocratic mixture of 3.5 millimolar (mM) Carbonate/1 mM Bicarbonate. Standard curves using known amounts of sodium nitrite, sodium nitrate, sodium lactate or sodium acetate solutions are generated and used for calibrating nitrate, nitrite, lactate and acetate concentrations.

### Example 1

### Contamination Prevention Using in a Single Feed Line Bioreactor System (Prophetic)

A single feed line bioreactor system (FIG. 1) is set up for continuous growth of Pseudomonas stutzeri strain BR5311 (ATCC PTA-11283) under anaerobic conditions. The nutrient feed reservoir (2) consists of a 50 ml sterile disposable syringe (Beckton Dickinson, Franklin Lakes, N.J.) mounted on a syringe pump (Harvard Apparatus, Holliston, Mass.) which delivers a growth medium containing inhibitory nutritional components into the feed line (3). The feed line first connects to a feed pump (4) prior to connecting to the bioreactor (6). The bioreactor is a sterile 1 liter glass Erlenmeyer flask with a side arm port (for example, part 6979, Ace Glass Incorporated, Vineland, N.J.). The feed line (3) enters the bioreactor through a tube going into the top of the Erlenmeyer flask and ends below the liquid surface so as to introduce the nutrients below the surface of the liquid. A sterile magnetic stirrer is used to agitate the flask (part 13521, Ace Glass Inc.) where feed line (3) connects. The side arm port (7) fixes the level of the liquid in the Erlenmeyer flask by providing an overflow exit for excess liquid out of the bio reactor.

The starter culture for the bioreactor is prepared by adding an inoculum (0.1 ml) of Pseudomonas stutzeri cells to 500 ml of the inoculum growth medium for Pseudomonas stutzeri as described above, and is placed into the bioreactor under anaerobic conditions. Following an overnight growth period, a cell culture with a density of approximately 10⁷ cells per milliliter (cells/ml) is obtained. The volume of the medium and cells that is added to the bioreactor is such that the liquid level in the bioreactor is at the level of the side arm port (7) which is higher than the end point of the nutrient feed tube that is below the liquid level. Consequently, any addition of fluid to the bioreactor will result in excess fluid flowing out the side port. Analysis of the inoculum growth medium in the bioreactor after 24 hours shows that half the sodium nitrate (0.5 g) is consumed in one day. At this point, the growth medium containing inhibitory nutrient components (Inhibitory growth medium, above) from the nutrient feed reservoir (2) is fed continuously at a rate of 2.41 grams/day through the feed line and into the bioreactor. This rate matches the specific nitrate consumption rate observed during the first day of growth. After three days of continuous feeding of the growth medium with inhibitory nutritional components, the number of cells in the bioreactor increases to about 10⁷-10⁸ cells/ml, and periodic testing (checking growth on LB agar plates and most probable numbers count, well known in the art) shows that this population is maintained over the course of 25 days that the bioreactor is operated and the growth medium with inhibitory nutritional components is continuously fed into the bioreactor.

Pseudomonas stutzeri is a motile organism and forms biofilms. Either mechanism, motility or biofilm formation, could allow back growth up the feed line and into the nutrient feed reservoir over the extended period of the bioreactor operation in the absence of inhibitory growth medium. Visual inspection indicates that the walls of the nutrient feed reservoir are clean, i.e. no biofilm is observed.

Results of analysis of the wt % of nitrate, nitrite and the growth substrate (acetate) present in the growth medium with inhibitory nutritional components in the nutrient feed reservoir (2) and the bioreactor effluent (7) show that no nitrate or acetate is consumed in the nutrient feed reservoir (2) thus indicating lack of contamination of the nutrient feed reservoir. Additionally, analysis of the bioreactor effluent (7) shows that the limiting component acetate is not present. Thus application of the growth medium containing the inhibitory nutritional components prevents cells growing in the bioreactor from back growth in feed line (3c) and contaminating the feed line and the nutrient feed reservoir.

What is claimed is:

1. A method for preventing contamination of a feed line and nutrient feed reservoir in a bioreactor system, wherein the bioreactor system comprises a bioreactor, at least one nutrient feed reservoir, and at least one feed line that connects a nutrient feed reservoir to the bioreactor, wherein the method comprises the steps of:
a) adding to the bioreactor an initial charge of cells and inoculum growth medium;
b) filling the feed line with a second growth medium, comprising one or more inhibitory nutritional component, from the nutrient feed reservoir; and
c) adding the second growth medium in the feed line of (b) to the bioreactor of (a) forming a growth medium mixture in the bioreactor;
whereby the inhibitory nutritional component of the second growth medium is diluted in the growth medium mixture in the bioreactor and wherein the cells of (a) grow.

2. The method of claim 1 wherein the second growth medium containing one or more inhibitory nutritional component prevents contamination of the nutrient feed line and the nutrient feed reservoir.

3. The method of claim 2 wherein the inhibitory nutritional component has a concentration in the second growth medium that is greater than the concentration that is minimally inhibitory for growth of the cells in the bioreactor.

4. The method of claim 1 wherein the concentration of the inhibitory nutritional component of the second growth medium is below the minimally inhibitory level in the growth medium mixture in the bioreactor.

5. The method of claim 3 wherein the concentration in the growth medium mixture in the bioreactor of the inhibitory nutritional component of the second growth medium is less than about 10% of the concentration of the inhibitory nutritional component in the second growth medium.

6. The method of claim 1 wherein the second growth medium has an acetate concentration that is greater than 1%.

7. The method of claim 6 wherein the acetate concentration in the second growth medium in the nutrient feed reservoir remains constant.

8. The method of claim 1 wherein the second growth medium has a lactate concentration that is greater than 1%.

9. The method of claim 8 wherein the lactate concentration in the second growth medium in the nutrient feed reservoir remains constant.

10. The method of claim 1 wherein the second growth medium has a nitrate concentration that is greater than 1%.

11. The method of claim 10 wherein the nitrate concentration in the second growth medium in the nutrient feed reservoir remains constant.

12. The method of claim 1 wherein the bioreactor system lacks any contamination preventing intervening physical devices in either the nutrient feed reservoir or the feed line.

13. The method of claim 1 wherein the cells of (a) are cells having properties of motility, biofilm formation, or both motility and biofilm formation.

14. A bioreactor system comprising a bioreactor, at least one nutrient feed reservoir containing a second growth medium comprising one or more inhibitory nutritional component, and at least one feed line containing said second growth medium that connects the nutrient feed reservoir to the bioreactor.
